(19) United States
(12) Patent Application Publication
(10) Pub. No.: US 2004/0137553 A1
(43) Pub. Date: Jul. 15, 2004

(54) Enzymic microassay for measurement of chlorite, chlorate and perchlorate

(75) Inventors: John D. Coates, Walnut Creek, CA (US); Susan O'Connor, Walnut Creek, CA (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701-3271, US

(73) Assignee: Southern Illinois University

(21) Appl. No.: 10/457,836

(22) Filed: Jun. 9, 2003

Related U.S. Application Data
(60) Provisional application No. 60/386,894, filed on Jun. 7, 2002.

(51) Int. Cl. C12Q 1/28

(52) U.S. Cl. 435/28

(57) ABSTRACT

An enzymatic assay for chlorite, chlorate and perchlorate determination based on horseradish peroxidase, chlorate reductase, or nitrate reductase and a reacting dye was developed. The assay is quick and simple requiring less than 30 min; is cheap costing less than $0.05 per sample; uses standard laboratory equipment; is sensitive to a lower limit of 0.4 micromolar and linear up to 6.0 millimolar; is highly reproducible and interference free. The application of this assay to chlorite analysis in treated water will significantly reduce one of the major costs associated with the use of chlorine dioxide as a disinfectant and improve the drinking water quality.

### CROSS-REFERENCE TO RELATED APPLICATION

This application is related to, and claims a benefit of priority under 35 U.S.C. § 119(e) and/or 35 U.S.C. § 120 from, copending U.S. Serial. No. 60/386,894, filed Jun. 7, 2002, the entire contents of which are hereby expressly incorporated by reference for all purposes.

### BACKGROUND OF THE INVENTION

The government owns rights in the present invention pursuant to grant number DACA72-00-C-0016 from the US Department of Defense.

1. Field of the Invention

The present invention relates generally to the fields analytical chemistry and enzymology. More particularly, it concerns a new, rapid calorimetric assay for the detection of chlorite in environmental samples.

2. Description of Related Art

In the last two decades it has become apparent that the treatment of drinking water with chlorine can result in the abiotic production of potentially toxic tribalomethanes (Rook, 1974; Bellar et al., 1974), some of which, such as chloroform, are known to be carcinogenic (NC1, 1976). As a result, the use of chlorine dioxide for potable and waste water treatment has been proposed and it is rapidly becoming the disinfectant of choice amongst the drinking water providers throughout the U.S., Europe, Canada, and Japan. Current estimated annual production of chlorine dioxide to satisfy this demand is approximately 1.7 million metric tonnes (D. J. Denby, Sterling Pulp Chemicals personal communication). Similarly to hypochlorite, chlorine dioxide is a strong disinfectant over a wide pH range due to its strong oxidizing potential. It is effective in killing bacteria and deactivating viruses (Narkis et al., 1988; Narkis et al., 1989; Narkis and Kott, 1992). In addition, it is not known to form any tribalomethanes (Weinberg and Narkis, 1992) and has been shown to significantly reduce odor and color in finished water (Gordon and Tachiyashiki, 1991).

However, the use of chlorine dioxide is carefully regulated due to the toxicity of its active ingredient sodium chlorite (NaClO₂) and it must be registered by various government organizations. In the United States, these may include EPA, FIFRA, FDA or USDA. Chlorite is regulated under stage 1 of the Disinfectant/Disinfection Byproduct Rule of the 1996 Safe Drinking Water Act Amendments and continuous analysis for chlorite in treated water poses a prohibitive cost to smaller treatment facilities thus preventing their adoption of the superior disinfectant qualities of chlorine dioxide. This, and other guidelines are instituted because of the toxicity of chlorite to animals. Exposure to excessive dosages can severely damage organs and reduce the cellular and blood level of glutathione which serves to protect cells from oxidizing agents. At concentrations at or above 100 mgL⁻¹ chlorite causes a decrease in red blood cell count, hemoglobin concentration and packed cell volume after a 30 day exposure (Couri et al., 1982). A Maximum Contaminant Level (MCL) for chlorite has been established at 1 mgL⁻¹. As part of this, the EPA published a Direct Final Rule that approves the use of updated versions of several ASTM, Standard Methods, and Department of Energy analytical methods for compliance monitoring of certain drinking water contaminants.

Currently the recognized method for reliable chlorite determination is the use of ion chromatographic analysis with conductivity detection. However, this is an extremely costly and time-consuming method that requires specialized-equipment and trained personnel. As such, the adoption of chlorine dioxide as a disinfectant of choice by the Municipal Water treatment industry for drinking water supplies is hesitant although it offers significant advantages over alternative disinfectants.

Therefore, it would be beneficial to provide a method for reliable chlorite determination that is quick, simple, and cheap. Preferably, this method should use standard laboratory equipment, have a broad linear working range for chlorite detection, be, reproducible, essentially interference free, and use a minimal amount of sample.

### SUMMARY OF THE INVENTION

In accordance with the present invention, there is provided an assay for determining the presence of chlorite, chlorate and perchlorate in a sample comprising: (a) providing a sample; (b) contacting said sample with horseradish peroxidase (HRP) a calorimetric dye; (c) assessing color transformation, wherein color change indicates the presence of chlorite in said sample mixture. The assay may be solid phase, using horseradish peroxidase fixed to a solid support, or it may be conducted in solution. The volume of the sample mixture is about 10 μl-5 ml. The time from step (b) to step (c) is about 5-45 min. Step (b) in the assay is performed at a temperature of about 20° C.-30° C. or in some examples the temperature is about 25° C. The assay also comprises performing a positive control reaction and a negative control reaction.

The assay method can be applied towards homogeneous or heterogeneous samples. Samples can also be solutions. The samples can be from soil, water, sediment or wastestream. Where chlorate/perchlorate is to be measured, the assay is adapted by the addition of perchlorate reductase or nitrate reductase.

The colorimetric dye used in the assay method can be o-dianisidine, lissamine green, methyl viologen, or similar dye which chemically reacts with chlorine dioxide to give a measurable color change. The solid assays can be semi-quantitative. However, the liquid assay is highly quantitative. The detection range for the solid and liquid assay can be about 0.4 μm to 6000 μm. Sample mixture is approximately at pH 7.0-7.5 for the assay. The solid phase assay support can be a column, a bead, or a dipstick which can be comprised of paper, plastic, diamatacious earth or alginate.

The present invention also provides kits for determining the presence of chlorite, chlorate and perchlorate in a solid or liquid sample. The kits are comprised of horseradish peroxidase, or perchlorate reductase or nitrate reductase, a calorimetric dye, and optionally a cuvette or a solid support such as a dipstick, and/or additional agents in a suitable container. The kits may also comprise suitably aliquoted standards for chlorite determination and/or additional agent compositions of the present invention, whether labeled or unlabeled, as may be used to prepare a standard curve for a detection assay.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Dip-stick version of chlorite assay. Varying concentrations of chlorite from 0.001 mM to 5.0 mM can be seen.

FIGS. 2A-2B: FIG. 2A (increasing line) depicts the production of chlorine dioxide after the addition of chlorite to HRP. FIG. 2B depicts the production of a yellow product (ex. chlorine dioxide and o-dianisidine complex) in the same solution after HRP was inactivated and o-dianisidine was added. FIG. 2A (decreasing line) also shows the decreased absorbance at 395 nm indicating a decrease of chlorine dioxide after addition of the dye.

FIG. 3: The absorbance at 450 nm is shown for various concentrations of sodium chlorite for solutions with pH of 4.5, 5.0, 5.5, 6.0, 6.5, 7.0, and 7.5.

FIG. 4: The absorbance at 450 nm is shown for various concentrations of sodium chlorite for solutions with pH of 7.0, 7.1, 7.2, 7.3, 7.4, and 7.5.

FIGS. 5A-5B: The absorbance at 450 nm is shown for various concentrations of sodium chlorite for solutions having an incubation time of 5, 10, 15, 20, 30, 40, and 50 minutes. The temperature of the assay was 25° (FIG. 5A) and 37° C. (FIG. 5B).

FIG. 6: The reproducibility in absorbance measurements at 450 nm is shown for various concentrations of sodium chlorite by a triplicate assay of three separate weights.

FIG. 7: The absorbance at 450 nm is shown for 2 mM sodium chlorite solutions which also contain varying concentrations of sodium sulfate, sodium dithionite, sodium sulfide and sodium thiosulfate.

FIG. 8: The absorbance at 450 nm is shown for 2 mM sodium chlorite solution containing varying concentrations of sodium chlorate.

FIG. 9: The absorbance at 450 nm is shown for 2 mM sodium chlorite solution containing varying concentrations of nitrite and nitrate.

FIG. 10: The absorbance at 450 nm is shown for 2 mM sodium chlorite solution containing varying concentrations of sodium chloride.

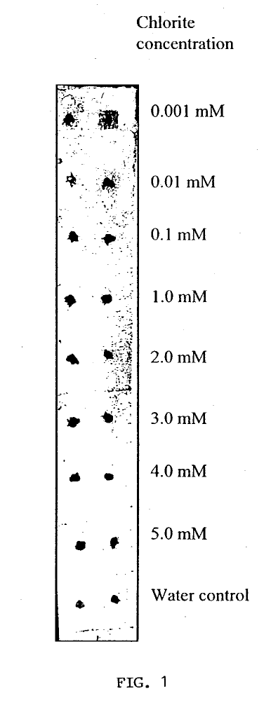

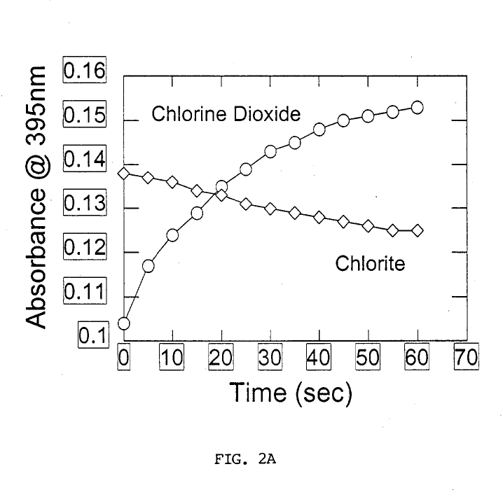

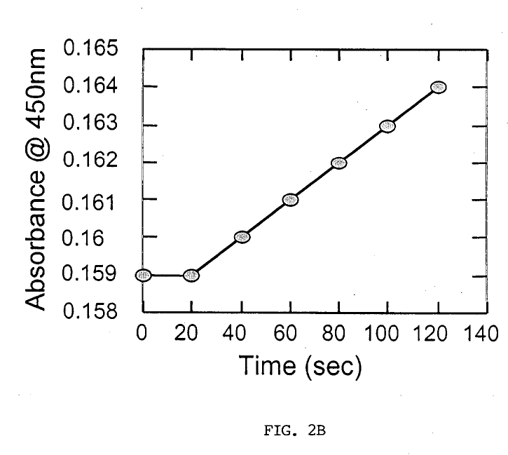

### DESCRIPTION OF ILLUSTRATIVE EMBODIMENTS

1. The Present Invention

A calorimetric assay for the determination of low level concentrations of chlorite, chlorate and perchlorate has been developed and is presented herein. The assay is based upon the enzymatic reaction between a reductase, horseradish peroxidase and chlorite, chlorate and perchlorate to form respectively chorine dioxide and chlorite. The chlorine dioxide formed then reacts with a colorimetric dye such as o-dianisidine or lissamine green to form a colored complex. The ensuing color formed is directly proportional to the initial chlorite concentration. In the case of the o-dianisidine a yellow product is produced which can be determined spectrophotometrically at 450 nm. This assay has been optimized for pH, temperature and incubation time (pH 7.3 for 20 min, at 25° C.). The assay is sensitive to chlorite concentrations as low as 0.4 micromolar and is linear and reproducible up to 6.0 millimolar. The assay does not suffer interference from common ions such as nitrate, nitrite, sulfate, ferric iron, bromate, or from other oxyanions of chlorine namely perchlorate and chlorate. This assay is accurate, reproducible, and sensitive, and does not require the use of specialized equipment or extensive training. It can be applied in one of two forms, either as a quantitative calorimetric assay or as a semi-quantitative dipstick.

This assay may be used for analyzing a wide variety of samples, such as a solution from soil, water, sediment or a wastestream. The sample may be taken directly from the source, solubilized in a water or buffer solution, or processed further before analysis. The volume of sample used will be about 10 μl-5 ml.

The EPA has set a limit of 1 ppm for chlorite which is 15 micromolar chlorite. This is detectable with the assay described herein.

2. Chlorite Reduction

The assay of the current invention is based on the enzymatic reaction between chlorite and horseradish peroxidase (HRP). Data suggests that the chlorite is converted to chlorine dioxide and chloride which is comparable to previously reported results. Enzymes other than HRP that are capable of converting chlorite to chlorine dioxide may also be used in the current invention. Both chlorate and nitrate reductases quantitatively convert chlorate/perchlorate to chlorite so they can not be used for the measurement of chlorite.

The HRP or other enzyme may be purified before use or used as obtained. Generally, “purified” will refer to a specific enzyme or other biomolecule that has been subjected to fractionation to remove various other proteins, polypeptides, or peptides, and which composition substantially retains its activity, as may be assessed, for example, by a protein assays or as would be known to one of ordinary skill in the art for the desired enzyme.

The HRP may also be substantially purified. Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition. The activity of HRP may also be varied, depending on the source, (e.g. Type I HRP has a lower activity and may be used). Concentrations of the HRP will be varied accordingly.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. Various techniques are suitable for use in protein purification will be well known to those of skill in the art. These include, for example, precipitation with ammonium sulfate, PEG, antibodies and the like or by heat denaturation, followed by centrifugation; chromatography steps such as ion exchange, ultrafiltration, dialysis, gel filtration, reverse phase, hydroxylapatite, lectin affinity and other affinity chromatography steps; isoelectric focusing; gel electrophoresis; and combinations of such and other techniques.

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

3. Colorimetric Dyes

Conventional colorimetric assays are advantageous in that they allow for direct, measurements of the color change. Absorbance measurements of color formation are simple, rapid, and can have a high range of linearity.

Colorimetric dyes that may be used with HRP include o-dianisidine, lissamine green, methyl viologen, 5-aminosalicylic acid (5AS), 2-2′ azino-di-(3-ethylbenzidine sulfonate (ABTS), o-phenylenediamine dihydrochloride (OPD), 3,3′, 5-5′-tetramethylbenzidine 9TMB), N-(4-amino-5-methoxy-2-methylphenyl)benzamide (AMMB), or N-acetyl-3,7-dihydroxyphenoxazine (Amplex Red) (Stewart et al., 2000).

Other dyes known in the art may be used if they chemically react with chlorine dioxide to give a measurable color change.

The wavelength for detection of the chlorite in the assay will be determined by the maximum absorbance of the calorimetric dye chosen, and can be found by scanning a spectrometer over the applicable range and recording the absorbance readings to determine the maxima.

4. Solid Phase Assays

In one aspect of the invention, a solid phase assay will be used in place of a cuvette and a solution phase assay. There are various supports for solid phase assays that can be used to determine the presence of chlorite in a sample. The solid phase materials are known from the art of separation science and chromatography. There are many kinds of chromatographic solid supports which may be used in the present invention. Examples of solid phase materials that may be used in the current invention include a column, beads, or a dipstick. Various materials may be used, e.g., paper, silica, hydroxyethyl cellulose, agarose, polyacrylamide, agarose-acrylamide, polyacrylamide, other plastics, diamatacious earth, alginate and the like (Sambrook et al., 1989). The support may be modified for binding of horseradish peroxidase or other enzyme useful in the current invention.

Generally, the specific support, pH, temperature and other conditions are selected to maximize the chlorite detection and linearity within the particular conformation. The support may be a dipstick, a plate, a column, a microcolumn, a cuvette, or the like. The color change may be detected by using a spectrophotometer or by visualization method under a suitable light source—the wavelength (i.e., UV, visible, or IR) is determined by the dye used.

5. Kits

Any of the compositions described herein may be comprised in a kit. In a non-limiting example, horseradish peroxidase, a calorimetric dye, and optionally a cuvette or a solid support such as a dipstick, and/or additional agent, may be comprised in a kit. The kits will thus comprise, in suitable container means, horseradish peroxidase, a colorimetric dye, a support material and/or an additional agent of the present invention.

The kits may also comprise suitably aliquoted standards for chlorite determination and/or additional agent compositions of the present invention, whether labeled or unlabeled, as may be used to prepare a standard curve for a detection assay. The kit may comprise positive and negative controls. The components of the kits may be packaged either in aqueous media or other buffered solution. The container means of the kits will generally include at least one dipstick, column, plate, cuvette, vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial or cuvette. The kits of the present invention also will typically include a means for containing horseradish peroxidase, a calorimetric dye, an optional cuvette or support material, additional agent such as standards, positive controls and negative controls, and any other reagent containers in close confinement for commercial sale. Such containers may include injection or blow-molded plastic containers into which the desired vials are retained. The kit may have a single container means, and/or it may have distinct container means for each compound.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one dipstick, column, plate, cuvette, vial, test tube, flask, bottle, syringe and/or other container means, into which the horseradish peroxidase or other enzyme and the dye are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

6. Definitions

As used herein, the term sample refers to a solution containing a known or unknown quantity of chlorite. The sample may be an aqueous solution, a suspension, or other solution. The sample may be comprised of soil, sediment or wastestream effluent.

As used herein, the term semi-quantitative refers to an assay that is able to provide relative concentrations of one or more substituants in a sample. Semi-quantitative techniques can be used to determine quantitative values by means of standards.

As used herein the specification, “a” or “an” may mean one or more. As used herein in the claim(s), when used in conjunction with the word “comprising”, the words “a” or “an” may mean one or more than one. As used herein “another” may mean at least a second or more.

### 7. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Sample Solution Phase Chlorite Assay

Procedure for a 10 μL Assay. To prepare the HRP/dye mix, a 100 mM phosphate buffer with a pH 7.3 is made by combining 26.6 mL 1 M KH₂PO₄(monobasic), 73.4 mL 1M K₂HPO₄ (Dibasic) and is make up to 1 L with nanopure water and check the pH, it should be 7.3. This is stored in a cold room. Stock HRP (30 U/mL) is made by weighting out 3 mg HRP (stored in −20° C.) and gently mixing in 10 mL 100 mM phosphate buffer. This solution is gently mixed bu not shaken. The HRP/Dye mix is made by diluting the stock to 1 U/mL in desired volume of 100 mM phosphate buffer and adding o-dianisdine to a final concentration of 0.128 mM. This dye is stored in a cold room and typically one would add 1.6 mL dye to 100 mL of dilute HRP. It is stable for 1 month if stored in the cold room and stored in the dark.

A stock solution of sodium chlorite, usually 20 mM, in water (2.26 mg/mL) is prepared and dissolve up just before use. A standard curve with stock chlorite in 0-5 mM range with final volume of 10 μL can be prepared. 10 μL of sample to be tested or chlorite standard is combined with 2 mL of HRP/Dye and left at room temperature for 20 min. The absorbance is read at 450 nm. If chlorite is present, there is a yellowish color.

Procedure for 2 mL Assay. For a larger volume assay, a 10 U/mL solution of HRP in 100 mM phosphate buffer is prepared at pH 7.3. Chlorite standard or sample (1.8 mL) is added with 32 μL of 8 mM O-dianisdine. Then 200 μL of 10 U/mL HRP solution is added and left for 20 min at room temperature. The absorbance is read at 450 nm.

### Example 2

### Solution-Phase Assay Set-Up

In one example, the assay is set up by taking 10 μL of a solution containing chlorite and add 2 mL of HRP/Dye solution and incubate at room temperature for 20 min. The optical density of the sample is read at 450 nm using a spectrophotometer. Unknown samples are determined from a standard curve prepared in the range of 0.02 nM to 5.0 nM chlorite.

In the first step, chlorite was added to HRP the production of chlorine dioxide was followed spectrophotometrically at 395 nm. This solution was then heat treated to inactivate HRP. Next, on addition of o-dianisidine to the heat treated solution, a yellow product developed within 20 secs (measured at 450 nm) and was accompanied by a concomitant decrease in absorbance at 395 nm indicating that the dye reacts abiotically with the chlorine dioxide.

### Example 3

### Sample Solid-Phase Chlorite Assay

To a dip-stick containing horseradish peroxidase and a colorimetric dye along with an area for a water control sample varying standard concentrations of chlorite are added. The dip-stick is then dipped into a sample solution letting all areas of the dip-stick be exposed to the solution except the water control area. Next the dipstick is incubated at room temperature for 20 minutes. The dipstick can then be read visually or by use of a spectrophotometer to determine the concentration of chlorite present in the sample solution.

### Example 4

### Optimization of Assay Conditions

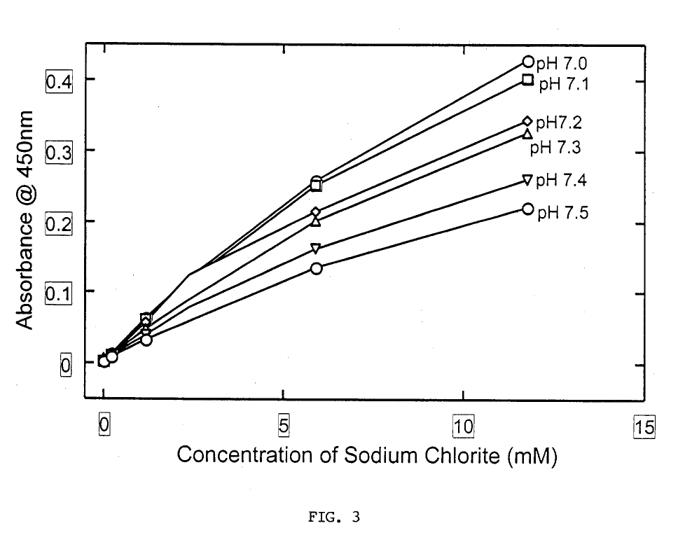

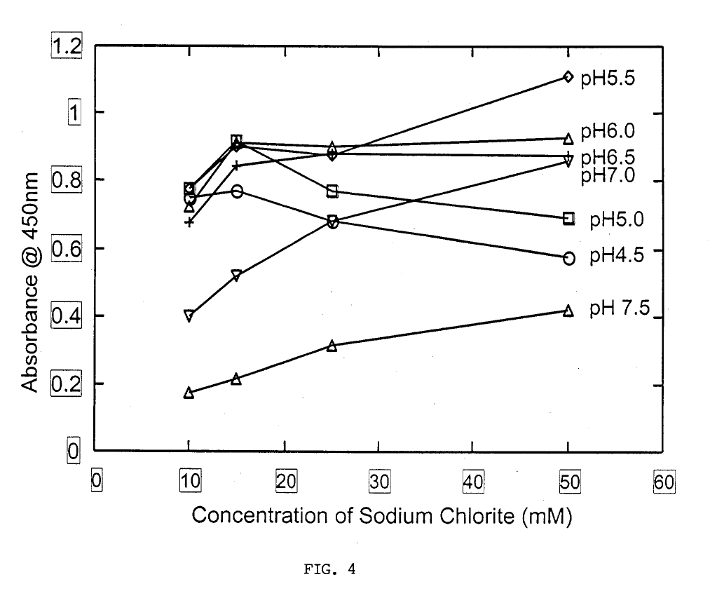

The optimum pH for the chlorite assay was initially assessed using a broad range of pH's as shown in FIG. 3. A 100 mM sodium acetate buffer was used for pH 4.5 to 5.5 and a 100 mM potassium phosphate buffer for pH's 6.0 to 7.5. The optimum pH range which exhibited the best linearity was pH 7.0-7.5 (FIG. 4). The pH of the assay buffer (100 mM phosphate) had a significant effect on color development. The optimum pH selected for the assay was pH 7.3 as it gave the most linear curve over the concentration range tested.

For other assay conditions, this optimization procedure may be repeated, and a different linear range may be found. For example, if a tris buffer is used, the optimal pH range will most be between 7.5 and 8.5.

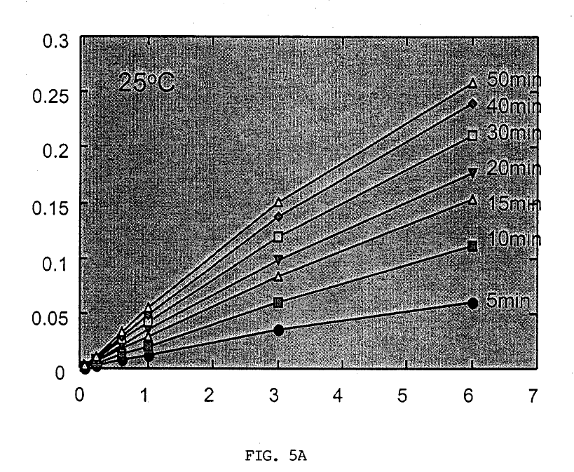

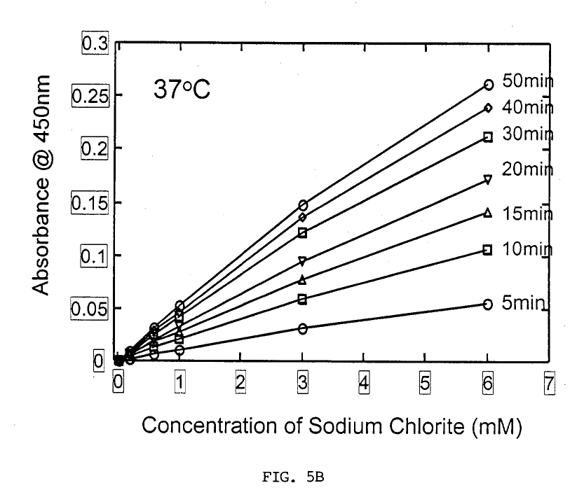

The chlorite assay was optimized for time and temperature. The assay was conducted at 37° C. (FIG. 5A) and 25° C. (FIG. 5B) and optical density was read every 5 to 10 min. Analysis of FIGS. 5A-5B shows that there was no obvious difference between the two temperatures selected, therefore, the preferred temperature for the assay varies from about 20° C.-40° C. The optimum time for incubation was 20 min. After the 20 min time, the linearity of the curve suffered although OD continued to increase (FIGS. 5A-5B). Therefore, the reaction time for the chlorate reduction is preferentially about 5-45 minutes, and is more preferably about 20 min for the current embodiment, however, shorter or longer times may be appropriate depending on pH, sample concentrations, and other conditions.

In addition to the reaction time, a holding time between collection of the sample and spectrometric detection to allow for transport of the sample to a lab or to collect a number of samples before analysis. It is preferred that the sample be kept cold by ice or other means during this time. If chilled, the sample should be allowed to warm up to room temperature before spectrometric detection because the HRP reaction is temperature dependent. The holding time may be up to 1, 2, 3, 4 or 5 hours.

It is also contemplated that a positive control reaction or a negative control reaction will be run with the assay reaction.

### Example 5

### Assay Characteristics

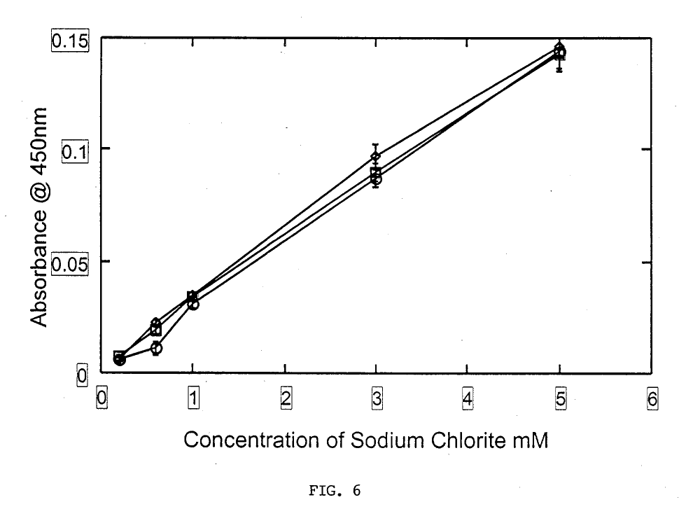

The chlorite assay is both linear and reproducible in the concentration range of 0.02-6.0 mM as shown in FIG. 6. This test was conducted with three separate weights of chlorite from which triplicate assays were performed.

### Example 6

### Effect of Contaminants

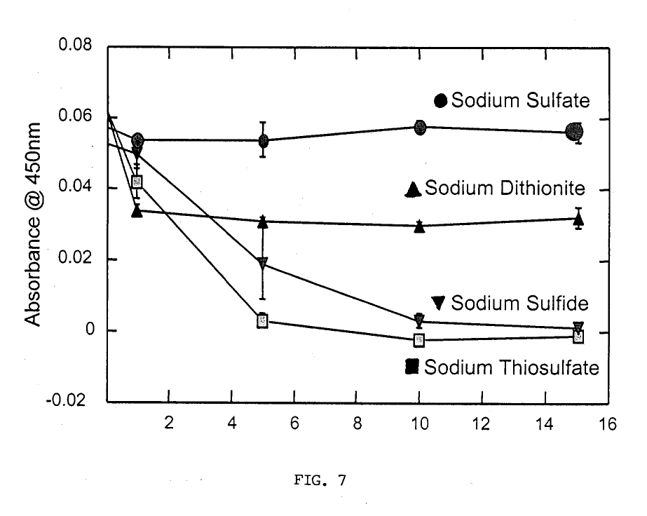

At a constant concentration of sodium chlorite (2 mM), the assay was not affected by the presence of sulfate, but was inhibited by reducing agents such as dithionite, sulfide and thiosulfate (FIG. 7). This is not unexpected since sulfide is a known inhibitor of HRP and so presumably is thiosulfate and dithionite.

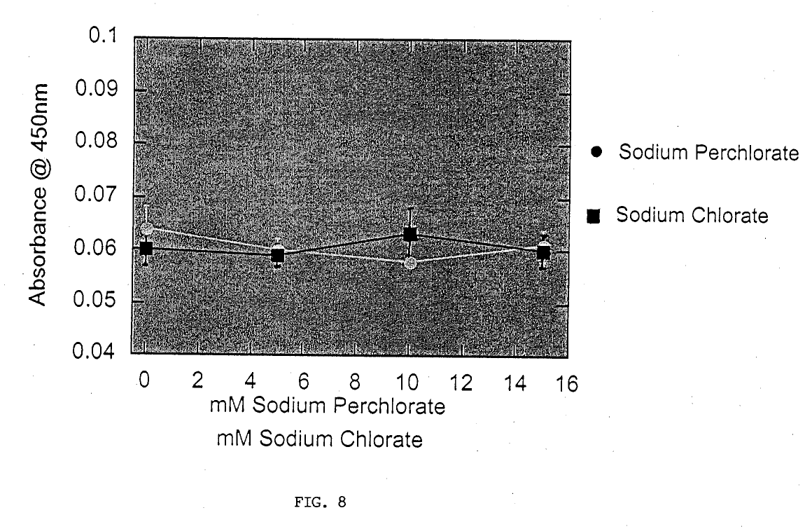

Furthermore, at a constant concentration of sodium chlorite (2 mM), the assay was not affected by the presence of other oxyanions of chlorine such as perchlorate or chlorate over a wide concentration range (1.0-15.0 mM, FIG. 8).

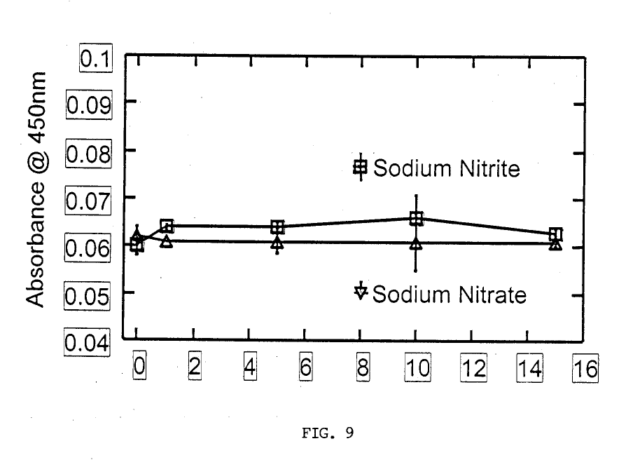

At a constant concentration of sodium chlorite (2 mM), the assay was not affected by the presence of nitrate or nitrate over a wide concentration range (1.0-15.0 mM, FIG. 9).

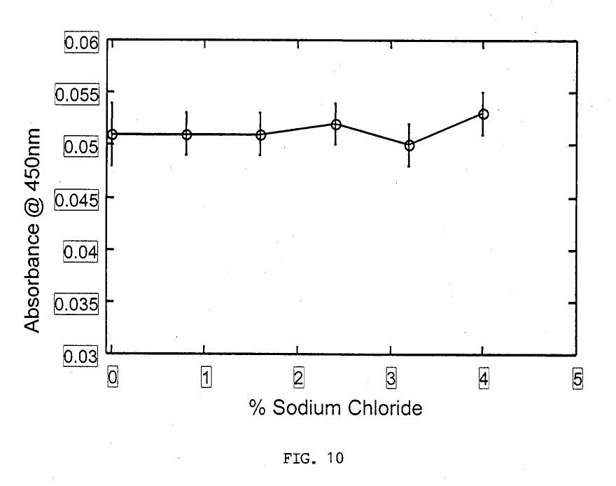

At a constant concentration of sodium chlorite (2 mM), the assay was not affected by the various concentrations of salt, as shown in FIG. 10. This indicates that the assay is compatible for use with marine samples.

### Example 7

### Applications

The chlorite assay has been successfully used by this lab during the purification of the enzyme chlorite dismutase from the (per)chlorate reducing micro-organism Dechloromonas agitata str. CKB Since the assay only requires a 10 μL sample, all fractions collected could be assayed for activity. Thus, the assay allows for the identification and selection of which fractions contain the highest activity and thus the highest concentration of active enzymes to purify. This resulted in a 55-fold purification of the enzyme with a specific activity of 1928 μmol chlorite dismutated per min per mg protein.

### Prophetic Example 8

### Field use of Chlorite Assay

This assay can be adapted for use in the field using hand held spectrophotometers. This will allow for rapid screening of areas for chlorite contamination and may be followed up with additional assays in a more sensitive laboratory setting if chlorite levels warrant further concern. General techniques for adapting assays for field use can be used. These techniques may include the optimization for smaller spectrophotometers or spectrophotometers with lower sensitivity, producing a kit for the rapid screening at a test site where highly trained clinicians are not required.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Bellar, Lichtenberg, Kroner, J. Am. Water Works Assoc., 66:703-706, 1974.

Couri, Abdel-Rahman, Bull, Environ. Health Perspect., 46:13-17, 1982.

Gordon and Tachiyashiki, Environ. Sci, Technol., 25:468-474, 1991.

Narkis and Kott, “Comparison between chlorine dioxide and chlorine for use as a disinfectant of wastewater effluents,” 1-1483-1492, Pergamon, Wash. DC, 1992.

Narkis, Offer, Betzer, “The use of chlorine dioxide in disinfecting of advanced treated effluents,” Tel-Aviv, 1988.

Narkis, Offer, Linenberg, Betzer, In: Water Chlorination-Chemistry, Environmental Impact and Health Effects, Jolley (Eds.), Lewis Pub Inc., 955-966, Chelsa, Mich., 1989.

NCI, Report on carcinogenesis bioassay of chloroform (Natl. Cancer Institute), 1976.

Rook, J. Water Treat. Exam., 23:234-236, 1974.

Sambrook et al., “Molecular Cloning,” A Laboratory Manual, 2d Ed., Cold Spring Harbor Laboratory Press, New York, 13.7-13.9:1989.

Stewart et al., Methods in Cell Science, 22:67-78, 2000.

Weinberg and Narkis, “Oxidation byproduct resulting from the interaction of chlorine dioxide with nonionic surfactants,” Vanderbilt Univ., TN, 1992.

What is claimed is:

1. An assay for determining the presence of chlorite, chlorate and perchlorate in a sample comprising:
(a) providing a sample;
(b) contacting the sample with horseradish peroxidase (HRP) and a colorimetric dye; and
(c) assessing color transformation, wherein color change indicates the presence of chlorite in the sample mixture.

2. The assay of claim 1, wherein the assay is a solid phase assay.

3. The assay of claim 2, wherein HRP is fixed to a solid support.

4. The assay of claim 1, wherein the assay is conducted in solution.

5. The assay of claim 1, wherein the volume of the sample mixture is about 10 μl-5 ml.

6. The assay of claim 1, wherein the time from step (b) to step (c) is about 5-45 min.

7. The assay of claim 1, wherein step (b) is performed at a temperature of about 20° C.-30° C.

8. The assay of claim 7, wherein step (b) is performed at a temperature of about 25° C.

9. The assay of claim 1, further comprising performing a positive control reaction.

10. The assay of claim 1, further comprising performing a negative control reaction.

11. The assay of claim 1, wherein the sample is homogeneous.

12. The assay of claim 1, wherein the sample is heterogeneous.

13. The assay of claim 1, wherein the sample is derived from soil, water, sediment or waste stream.

14. The assay of claim 1, wherein the colorimetric dye is o-dianisidine, lissamine green, methyl viologen, or a similar dye which chemically reacts with chlorine dioxide to give a measurable color change.

15. The assay of claim 2, wherein the solid assay is semi-quantitative.

16. The assay of claim 4, wherein the liquid assay is quantitative.

17. The assay of claim 1, wherein the detection range is about 0.4 μm to 6000 μm.

18. The assay of claim, wherein the sample mixture is at a pH of approximately 7.0-7.5 for the assay.

19. The assay of claim 3, wherein the solid phase support is a column, a bead, or a dipstick.

20. The assay of claim 19, wherein the dipstick is comprised of paper, plastic, diamatacious earth or alginate.

21. The assay of claim 1, wherein chlorate or perchlorate are measured, and step (b) further comprises contacting the sample with chlorate reductase or nitrate reductase.
